(19) United States
(12) Patent Application Publication
(10) Pub. No.: US 2025/0064729 A1
(43) Pub. Date: Feb. 27, 2025

(54) METHODS AND FORMULATIONS FOR IMPROVING THE PHARMACOKINETIC PROPERTIES OF NUTRACEUTICAL COMPOUNDS

(71) Applicant: UNIVERSITY OF SOUTH CAROLINA, COLUMBIA, SC (US)

(72) Inventor: DAPING FAN, COLUMBIA, SC (US)

(21) Appl. No.: 18/813,465

(22) Filed: Aug. 23, 2024

Related U.S. Application Data
(60) Provisional application No. 63/578,464, filed on Aug. 24, 2023.

(51) Int. Cl. A61K 9/06; A61K 31/12; A61K 31/122; A61K 31/352; A61K 47/54

(52) U.S. Cl. CPC A61K 9/06; A61K 31/12; A61K 31/122; A61K 31/352; A61K 47/544

(57) ABSTRACT

In general, the present disclosure is directed to compositions and methods for formulating thereof for oral administration. The composition may be in the form of a hydrogel. The hydrogel may include a phospholipid complex comprising a solubilized phospholipid and a solubilized nutraceutical compound.

### CROSS REFERENCE TO RELATED APPLICATION

This application claims filing benefit of U.S. Provisional Patent Application Ser. No. 63/578,464 having a filing date of Aug. 24, 2023, which is incorporated herein by reference for all purposes.

### GOVERNMENT SUPPORT CLAUSE

This invention was made with government support under grants R41AT009964, R43AT011171, and UT1AA030690 awarded by National Institutes of Health (NIH). The government has certain rights in the invention.

### BACKGROUND

Naturally occurring compounds are believed to harbor value in preventing or treating multiple diseases in in vitro and in vivo studies. While these compounds usually need to be administrated in a long term to achieve ideal preventive or therapeutic efficacy, oral administration is by far the easiest and most acceptable route for their delivery. However, their low water solubility and low oral bioavailability present a large obstacle in the development of these compounds as therapeutics.

In particular, naturally occurring nutraceutical compounds, such as quercetin, curcumin, or emodin, are promising candidates to be developed as therapeutic agents for various diseases such as cancer, cardiovascular disease, Alzheimer's disease, inflammatory bowel diseases, and fibrotic diseases. For instance, emodin has shown potential to inhibit inflammation in various settings. Emodin has been shown to attenuate the severity of experimental disease models including arthritis, liver damage, atherosclerosis, myocardial ischemia, and breast cancer. It has been reported that emodin can reduce breast tumor growth and metastasis in mouse models. However, they have not been approved by the U.S. Food and Drug Administration (FDA) for any clinical applications. This is largely due to their low water solubility and low bioavailability.

As such, there is a need in the art for compositions and methods for formulating said compositions that improve the pharmacokinetic properties of nutraceutical compounds that can be utilized to prevent and/or treat various diseases.

### SUMMARY

In general, the present disclosure is directed to compositions and methods for formulating thereof for oral administration. The composition may be in the form of a hydrogel. The hydrogel may include a phospholipid complex comprising a solubilized phospholipid and a solubilized nutraceutical compound.

Other features and aspects of the present disclosure are discussed in greater detail below.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates steps to formulate an emodin or quercetin emulsion with topcithin.

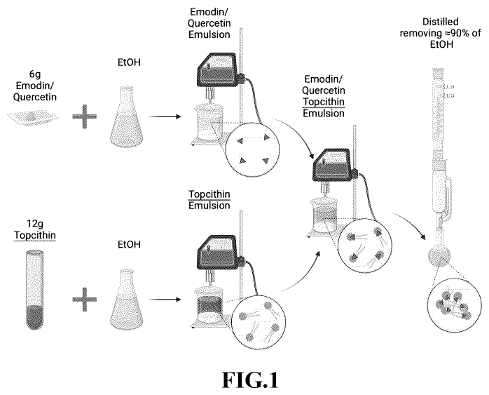

FIG. 2 schematically illustrates steps to formulate crosslinked matrix wet pellets.

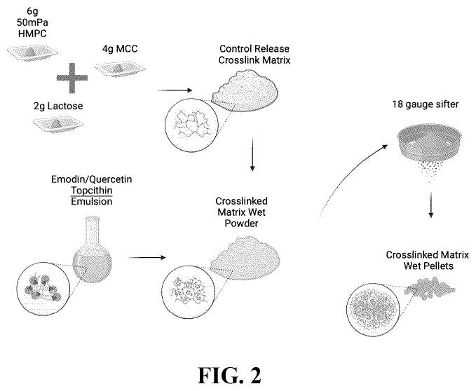

FIG. 3 schematically illustrates steps to formulate the AP capsule including a nutraceutical compound.

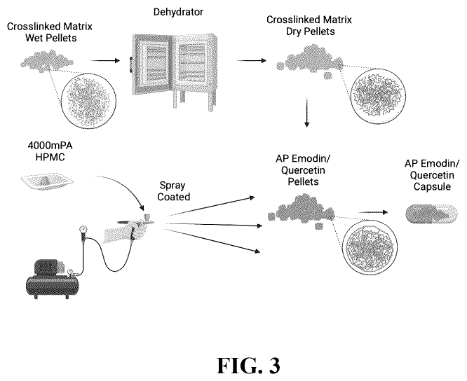

FIG. 4 schematically illustrates procedures for preparing the PHMHP formulation of a nutraceutical compound.

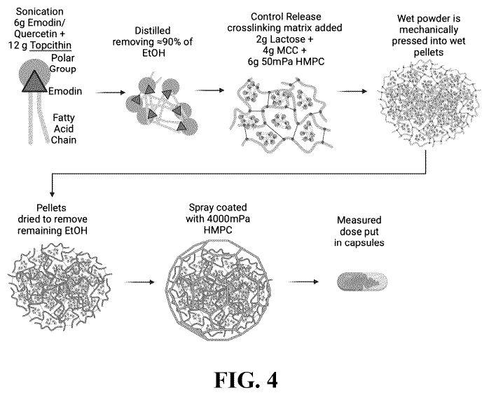

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is directed to pharmaceutical compositions and methods for preparation thereof that may be utilized in the treatment of various diseases. Advantageously, methods disclosed herein may improve pharmacokinetic properties and oral bioavailability of said pharmaceutical compositions.

In one example embodiment, the pharmaceutical composition may include a nutraceutical compound. “Nutraceutical compound” as used herein may include, but are not limited to, vitamins, minerals, amino acids, antioxidants, fatty acids, and phytochemicals (plant-derived chemicals) that may be useful in treating or reducing a subject's risk of developing chronic diseases or improving overall well-being. For instance, nutraceutical compounds disclosed herein may be useful in prevention or inhibition of tumorigenesis as well as in decreasing side effects of cancer chemotherapy. In another example embodiment, a “nutraceutical compound” may refer to a naturally occurring compound. For instance, a naturally occurring “nutraceutical compound” may be derived from antioxidants; omega-3 fatty acids; plants such as algae, aloe vera, seaweed, and wheatgrass; teas and herbs such as ginseng and Echinacea.

In one example embodiment, the nutraceutical compound may be a polyphenol flavonoid. For instance, the polyphenol flavonoid may be 3,3′,4′,5,7-pentahydroxylflavone (“quercetin”). Quercetin is a flavanol found in a variety of plants used as food, including apples, berries, capers, grapes, tea, and tomatoes. It is also found in many medicinal herbs, such as Ginkgo biloba, Hypericum perforatum, and Sambucus canadensis. As a medicinal compound, quercetin is known to have many biological activities, including anti-carcinogenic, anti-inflammatory, antiviral, and antioxidant activities. Quercetin is also believed to increase mental and physical performance and benefit mental health.

In one example embodiment, the nutraceutical compound may be emodin (1,3,8-trihydroxy-6-methylanthraquinone). Emodin is a natural trihydroxy-anthraquinone which is found in several Chinese herbs, including rhubarb (Rheum palmatum) and tuber fleece flower (Polygonum multiflorum, also commonly known as Chinese knotweed or he shou wu). Emodin harbors a variety of pharmacological properties, including anti-cancer, hepatic protection, anti-inflammatory, antimicrobial, and antioxidant activities. For instance, emodin has been shown to attenuate the severity of experimental disease models including arthritis, liver damage, atherosclerosis, myocardial ischemia, and breast cancer. Also, it has been reported that emodin may reduce breast tumor growth and metastasis in mouse models.

In order for the nutraceutical compound to be effectively utilized in a clinical therapy, it can be delivered so as to be provided with suitable bioavailability. For instance, as an in vivo treatment method, the pharmaceutical composition including the nutraceutical compound and a pharmaceutically compatible carrier can be delivered to a subject via any pharmaceutically acceptable delivery system. In general, the nutraceutical compound may be administered to a subject according to known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. Osmotic mini-pumps also may be used to provide controlled delivery of the nutraceutical compound through cannulae to the site of interest either systemically or locally, such as directly into a metastatic growth. In certain embodiments, the nutraceutical compound can be administered directly to the area of a tumor or cancer tissue, including administration directly to the tumor stroma during invasive procedures. The nutraceutical compound also may be placed on a solid support such as a sponge or gauze for administration.

Pharmaceutically acceptable carriers include, but are not limited to, saline, buffered saline, glucose in saline, etc. Solid supports, liposomes, nanoparticles, microparticles, nanospheres, or microspheres also may be used as carriers for administration of emodin. As used herein, the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The appropriate dosage (“therapeutically effective amount”) of the nutraceutical compound can depend, for example, on the severity and course of the cancer, whether the nutraceutical compound is administered for therapeutic purposes or in prevention of side effects of other chemotherapy agents, previous therapy, the patient's clinical history and response to the said nutraceutical compound, and the discretion of the attending physician, among other factors. For instance, emodin may be administered to a subject at one time or over a series of treatments and may be administered to the subject at any time.

In one example embodiment, a therapeutically effective amount of the nutraceutical compound may be in the range of from about 0.5 μg/mL to about 700 μg/mL, such as from about 5 μg/mL to about 600 μg/mL, such as from about 7 μg/mL to about 500 μg/mL, such as from about 10 μg/mL to about 400 μg/mL, such as from about 20 μg/mL to about 300 μg/mL, such as from about 50 μg/mL to about 250 μg/mL, or any range therebetween. As expected, the dosage will be dependent on the condition, size, age, and condition of the patient.

The nutraceutical compound may be administered, as appropriate or indicated, in a single dose as a bolus or by continuous infusion, or as multiple doses by bolus or by continuous infusion. Multiple doses may be administered, for example, multiple times per day, once daily, multiple times per weck, every 2, 3, 4, 5, 6, or 7 days, weekly, every 2, 3, 4, 5, or 6, weeks or monthly. However, other dosage regimens may be useful. The progress of this therapy may be monitored by conventional techniques.

It can be advantageous to formulate oral or parenteral compositions in dosage unit form for case of administration and uniformity of dosage. Dosage unit form as used herein includes physically discrete units suited as unitary dosages for the subject to be treated; each unit may contain a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

Pharmaceutical compositions for parenteral, intradermal, or subcutaneous injection can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (e.g., glycerol, propylene glycol, polyethylene glycol, and the like), carboxymethylcellulose and suitable mixtures thereof, vegetable oils (e.g., olive oil), and injectable organic esters such as ethyl oleate. A composition can contain minor amounts of auxiliary substances, such as wetting or emulsifying agents, pH buffering agents, and the like, that can enhance the effectiveness of the active ingredient. Proper fluidity may be maintained, for example, by the use of coating materials such as lecithin, by the maintenance of the required particle size in the case of dispersions and by the use of surfactants. A composition also may contain adjuvants such as preservatives, wetting agents, emulsifying agents, and dispersing agents. It also may be desirable to include isotonic agents such as sugars, sodium chloride, and the like.

For intravenous administration, suitable carriers include, without limitation, physiological saline, bacteriostatic water, Cremophor EL™ (BASF, Parsippany, N.J.), or phosphate buffered saline (PBS). In all cases, an injectable composition should be sterile and should be fluid to the extent that easy syringability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid, and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents and/or adjuvant materials can be included as part of an orally ingestible composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water; petroleum; oils of animal or plant origin such as peanut oil, mineral oil, soybean oil, or sesame oil; or synthetic oils may be added. A liquid form may further contain physiological saline solution, dextrose or other saccharide solution, or glycols such as ethylene glycol, propylene glycol, or polyethylene glycol. When administered in liquid form, a composition can contain from about 0.5 to 90% by weight the nutraceutical compound; in one embodiment, from about 1 to 50% by weight the nutraceutical compound.

For administration by inhalation, the nutraceutical compound may be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, a pharmaceutical composition can be formulated for sustained or controlled release of the nutraceutical compound. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially. Liposomal suspensions can also be used as pharmaceutically acceptable carriers. These can be prepared according to methods known to those skilled in the art.

In one example embodiment, sustained release of the nutraceutical compound can be provided through inclusion in the particles of a phospholipid carrier material. For example, particles can include a phospholipid carrier material in an amount up to about 90wt. %; for instance, from about 10 wt. % to about 60 wt. % in some embodiments. Examples of phospholipids can include, without limitation, phosphatidic acids, phosphatidylcholines, phosphatidylalkanolamines such as a phosphatidylethanolamines, phosphatidylglycerols, phosphatidylscrines, phosphatidylinositols, and combinations thereof. To impart a sustained release profile, the phase transition temperature of a specific phospholipid can be below, around, or above the expected physiological body temperature of a subject. In one embodiment, the phase transition temperature of a phospholipid carrier material can be from about 30° C. to about 50° C. (e.g., within about ±10° C. of the normal body temperature of patient). By selecting a phospholipid or a combination of phospholipids according to their phase transition temperature, the particles can be tailored to have controlled release properties. For example, by administering particles which include a phospholipid or combination of phospholipids that have a phase transition temperature higher than the patient's body temperature, the release of the nutraceutical compound may be slowed. On the other hand, rapid release can be obtained by including phospholipids having lower transition temperatures.

In one example embodiment, the nutraceutical compound may form a complex with pharmaceutical carriers or excipients that are suitable for oral administration. Such carrier materials may serve simply as bulking agents to control the concentration of the nutraceutical composition in the pharmaceutical composition, or may provide one or more alternate or additional functions to a pharmaceutical composition. For instance, a carrier material can enhance the stability of the pharmaceutical composition and/or can improve the dispersibility of the pharmaceutical composition within a dry powder.

Carrier materials may be amorphous, crystalline, or a combination of amorphous and crystalline. Exemplary carrier materials include, without limitation, carbohydrates, including monosaccharides (e.g., fructose, galactose, glucose, D-mannose, sorbose, and the like); disaccharides (e.g., lactose, trehalose, cellobiose, and the like); cyclodextrins (e.g., 2-hydroxypropyl-β-cyclodextrin); polysaccharides (e.g., raffinose, maltodextrins, dextrans, starch, and the like); celluloses (e.g., methyl cellulose, carboxymethylcellulose, hydroxypropyl cellulose, and the like); xanthan gum; carbomer; alginate; polyvinyl alcohol; acacia; chitosans; amino acids (e.g., glycine, arginine, aspartic acid, glutamic acid, cysteine, lysine, and the like); organic salts prepared from organic acids and bases (e.g., sodium citrate, sodium ascorbate, magnesium gluconate, sodium gluconate, tromethamine hydrochloride, and the like); peptides and proteins (e.g., aspartame, human serum albumin, gelatin, and the like); alditols (e.g., mannitol, xylitol, and the like); or combination of two or more different dry carrier materials.

In one example embodiment, the carrier material may be one or more biocompatible polymers. For instance, the biocompatible polymer may include, but is not limited to, a phospholipid or derivative thereof, cellulose or derivative thereof, or a combination thereof. In one embodiment, the phospholipid may be a solubilized using a suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. The solubilized phospholipid may be a lecithin, sorbitan monooleate, or acacia, or a combination thereof. For instance, the solubilized phospholipid may be a lecithin. “Lecithin” as used herein refers to a complex mixture of phospholipids (e.g., phosphatides) in combination with other substances (e.g., oils, triglycerides, glycolipids, sphingolipids, fatty acids, or carbohydrates). For instance, the lecithin may be soy lecithin, canola lecithin, sunflower lecithin, and/or safflower lecithin. In one embodiment, the lecithin may be a sunflower lecithin, which is a product that is commercially available under the trademark TOPCITHIN™.

In one example embodiment, the pharmaceutical composition may be delivered in the form of a hydrogel. For instance, in the hydrogel the nutraceutical compound disclosed herein may bind to the phospholipid via hydrogen bonding to form a phospholipid complex. Without wishing to be bound by theory, the nutraceutical compound's bioavailability may be improved by incorporating the nutraceutical compound into the phospholipid complex given the multi hydroxyl groups in the chemical structure of said nutraceutical compounds.

In one example embodiment, the hydrogel may include a crosslinked matrix that includes, without limitations, additional biocompatible polymers in conjunction with or alternative to a solubilized phospholipid. For instance, the hydrogel may include a cellulose polymer or a derivative of cellulose. Cellulose or a derivative of cellulose may include, but are not limited to, cellulose acetate, sodium carboxymethyl cellulose, ethylcellulose, nitrocellulose, bacterial cellulose, hydroxypropyl methylcellulose, etc. Cellulose is the most abundant polysaccharide, and it is inexpensive with good processibility, renewability, and case of physical and chemical modification. It has good mechanical properties, good hydrolytic stability, low toxicity, and excellent biocompatibility.

In one example embodiment, the hydrogel may include hydroxypropyl methylcellulose (HPMC; also known as, Hypromellose). HPMC is a hydrophilic (water soluble), biodegradable, and biocompatible polymer that is used in an array of applications including drug delivery.

In one example embodiment, the nutraceutical compound to phospholipid may be present in the composition at a ratio of from about 1:0.5 to about 1:10, such as from about 1:1 to about 1:7.5, such as from about 1:1 to about 1:5, or any range therebetween.

Methods disclosed herein involve a multiple step procedure to generate novel formulations for isolated, naturally occurring nutraceutical compounds derived from plants. Although these nutraceutical compounds are known to possess many health benefits for humans, the human body does not absorb and metabolize the chemical compounds adequately to obtain the health benefits when the chemical compounds are taken in their natural form. However, methods disclosed herein may, beneficially, be utilized to develop formulations that add complex layers to the chemical compounds, which causes the human body to absorb and metabolize the compounds in a way that increases their therapeutic benefit. Formulations disclosed herein are expected to significantly increase the oral bioavailability of nutraceutical compounds, such as emodin, quercetin, or curcumin.

The present disclosure may be better understood with reference to the following examples.

### EXAMPLES

### Materials and Methods

### Excipients and Chemicals

All chemicals were purchased from Spectrum Chemical (New Brunswick, NJ) or Sigma-Aldrich (St. Louis, MO) unless indicated otherwise. Pharmaceutical grade excipients were purchased from ColorCon (Harleysville, PA). Excipients and chemicals used include: microcrystalline cellulose (NF C1679), isopropyl alcohol, lactose monohydrate (NF LA106), PEG 8000, povidone K-30, povidone K-90, triethyl citrate, TCIC-137, simethicone, hypromellose, Topcithin (Sunflower lecithin, TOPCITHIN™ SF, Cargill, Netherlands), quercetin (Sigma-Aldrich), Emodin (Nanjing Langze), anhydrous calcium phosphate, Talc, magnesium stearate, hydroxypropyl methylcellulose (HPMC), absolute alcohol (Fisher Scientific), lactose Monohydrate, hypromellose 50 mPa-s, and hypromellose 4000 mPa-s.

### Preparation of PHMHP Formulations

Formulations of quercetin and emodin, AP-Quercetin, or AP-Emodin were prepared using the following steps:
1. Weigh 12 g topcithin (lecithin topcithin sunflower);
2. Use handheld sonicator to get top 6 g of topicithin to dissolve in 50 mL of ethanol;
3. Pour the dissolved portion of topicithin into a 500 ml beaker;
4. Use handheld sonicator to get remaining topicithin to dissolve in 50 mL of ethanol;
5. Pour the dissolved topicithin into the 500 ml beaker;
6. Steps 2-5 can be repeated until all topicithin is dissolved;
7. Add enough ethanol to get beaker to the 500 mL line;
8. Use handheld sonicator to more completely dissolve topithin in ethanol 250 mL for 60 minutes;
9. Put the topcithin solution in the bath sonicator and handheld homogenizer for 60 minutes;
10. Weigh 6 g emodin or quercetin;
11. Use a sonicator to completely dissolve emodin or quercetin in ethanol 250 mL in a 500 mL beaker;
12. Mix emodin or quercetin solution and topithin solution together in a 1000 mL beaker;
13. Add 50 mL to the two 500 mL beakers to wash any remaining residue and add that to the 1000 ml beaker until the level reaches 1000 mL;
14. Use handheld sonicator and handheld homogenizer together to more completely dissolve the mixture. This will take about 1 hour;
15. Mix emodin or quercetin solution and topithin solution completely in sonic water bath for 60 minutes;
16. Pour mixture into 1 L boiling round flask;
17. Connect 1 L flask to distiller;
18. Add ice to cool park of distiller;
19. Distill out ethanol at 65° C. until mixture begins sticking to wall of flask;
20. Weigh 2 g Lactose powder;
21. Weigh 4 g microcellulose powder;
22. Weigh 6 g 50 MpA HMPC powder;
23. Mix 2 g lactose and 4 g microcellulose, 6 g HMPC powder;
24. Mix liquid distillate with dry powder;
25. Press mixture until evenly mixed;
26. Hand press mixture through 18 mesh sieve to remove larger pellets and then a 20-mesh sieve to remove smaller pellets;
27. Transfer the remained pellets into a coating pan;
28. Dry the pellets at 60° C. and form hydrogel pellets by spraying 0.5% HPMC solution of high viscosity (in ethanol/water at a ratio of 50:50 (v/v));
29. Add adequate amount of lubricate ingredient to the resulting hydrogel pellets and then fill the pellets into size 000 capsules.

### Example 1

### Solubility Assessment

AP-Emodin and AP-Quercetin were determined to contain 20% (w/w) emodin and quercetin, respectively. The solubility of AP-Emodin and AP-Quercetin was determined under saturation conditions and compared with that of unformulated emodin or quercetin under the same conditions and in the following simulated gastrointestinal media: FaSSGF pH 1.6 (fasted-state simulated gastric fluid), FaSSIF pH 6.5 (fasted-state simulated intestinal fluid), and FeSSIF pH 5.0 (fed-state simulated intestinal fluid). Biological media (Biorelevant.com, London, UK) were prepared according to the manufacturer's instructions. To ensure that the emodin or quercetin concentrations in all samples were very similar, ˜20 mg of emodin or quercetin, and about 100 mg of AP-Emodin or AP-Quercetin were added to 10 ml of each simulated biological fluid. The resulting suspensions were left for 1 h at room temperature under constant magnetic stirring. A 200-μl aliquot of each suspension was taken into an Eppendorf tube and centrifuged at top speed, and 100 μl supernatant was taken for UV spectrometer detection at 434 nm for emodin and 371 nm for quercetin. The concentration was calculated based on standard curves established using pure emodin or quercetin completely dissolved in methanol. The solubilities are shown in Table 1. The data showed that our formulation significantly improved the solubility of emodin and quercetin by ˜3.4 fold in fed-state simulated intestinal fluid where in real life situation emodin and quercetin are absorbed from.

TABLE 1
Solubility of emodin and quercetin in simulated fluids

| | Emodin | Quercetin |
|---|---|---|
| Material | (ug/ml) | (ug/ml) |
| FaSSGF pH 1.6 | | |
| Emodin or quercetin | 7.1 | 20.8 |
| AP-Emodin or AP-Quercetin | 10.6 | 31.2 |
| FaSSIF pH 6.5 | | |
| Emodin or quercetin | 27.2 | 43.4 |
| AP-Emodin or AP-Quercetin | 38.3 | 106.3 |
| FeSSIF pH 5.0 | | |
| Emodin or quercetin | 113.6 | 145.5 |
| AP-Emodin or AP-Quercetin | 388.8 | 500.7 |

*FaSSGF fasted-state simulated gastric fluid, FaSSIF fasted-state simulated intestinal fluid, FeSSIF fed-state simulated intestinal fluid

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A composition comprising a hydrogel, the hydrogel comprising a phospholipid complex comprising a solubilized phospholipid and a solubilized nutraceutical compound.

2. The composition of claim 1, wherein the nutraceutical compound is hydrogen bonded to a phosphate group of the phospholipid.

3. The composition of claim 1, wherein the solubilized nutraceutical compound comprises emodin, quercetin, curcumin, or a combination thereof.

4. The composition of claim 1, wherein the solubilized nutraceutical compound comprises emodin.

5. The composition of claim 1, wherein the solubilized nutraceutical compound comprises quercetin.

6. The composition of claim 1, wherein the solubilized phospholipid comprises topicithin.

7. The composition of claim 1, further comprising a crosslinked matrix comprising a cellulose derivative.

8. The composition of claim 7, wherein the cellulose derivative comprises hydroxypropyl methylcellulose.

9. The composition of claim 1, wherein the composition is injectable.

10. The composition of claim 1, wherein the composition is in the form of a tablet, pill, capsule, troches, or a combination thereof.

11. A method for forming a hydrogel precursor, the method comprising:
contacting a phospholipid with a first solubilizing agent and contacting a nutraceutical compound with a second solubilizing agent;
combining the solubilized phospholipid and the solubilized nutraceutical compound to form the hydrogel comprising a phospholipid complex comprising the solubilized phospholipid and the solubilized nutraceutical compound.

12. The method of claim 11, wherein the nutraceutical compound is hydrogen bonded to a phosphate group of the phospholipid.

13. The method of claim 11, wherein the solubilized nutraceutical compound comprises emodin, quercetin, curcumin, or a combination thereof.

14. The method of claim 11, wherein the solubilized nutraceutical compound comprises emodin.

15. The method of claim 11, wherein the solubilized nutraceutical compound comprises quercetin.

16. The method of claim 11, wherein the solubilized phospholipid comprises topicithin.

17. The method of claim 11, the hydrogel precursor further comprising a crosslinked matrix comprising a cellulose derivative.

18. The method of claim 17, wherein the cellulose derivative comprises hydroxypropyl methylcellulose.

19. The method of claim 11, wherein the hydrogel precursor is injectable.

20. The method of claim 11, wherein the hydrogel precursor is in the form of a tablet, pill, capsule, troches, or a combination thereof.

21. The method of claim 11, wherein the first solubilizing agent and the second solubilizing agent are the same.

22. The method of claim 11, wherein the first solubilizing agent and the second solubilizing agent are different.
